(19) United States
(12) Patent Application Publication
(10) Pub. No.: US 2017/0312211 A1
(43) Pub. Date: Nov. 2, 2017

(54) APPLICATOR FOR AN ORAL CARE COMPOSITION

(71) Applicant: KONINKLIJKE PHILIPS N.V., EINDHOVEN (NL)

(72) Inventor: Bart GOTTENBOS, Budel (NL)

(21) Appl. No.: 15/513,314

(22) PCT Filed: Sep. 22, 2015
(86) PCT No.: PCT/EP2015/071626

§ 371 Date: Mar. 22, 2017

(30) Foreign Application Priority Data
Sep. 26, 2014 (EP) 14186696.2

(51) Int. Cl. A61K 8/73; B65D 35/14; A61Q 11/00

(52) U.S. Cl. CPC A61K 8/73; A61Q 11/00; B65D 35/14

(57) ABSTRACT

Disclosed is a new system for the application of mucoadhesive oral care compositions. The system comprises an applicator, the inner surface of which is made anti-adhesive. Particularly, said inner surface is rendered positively charged, so as to be anti-adhesive for positively charged mucoadhesive oral care compositions, such as those based on chitosan.

### FIELD OF THE INVENTION

The invention is in the field of oral care, and pertains to a system for the administration of oral care active agents. Particularly, the invention pertains to an applicator for adhesive particles, e.g., gel particles.

### BACKGROUND OF THE INVENTION

The human oral cavity, notably teeth and gums, is generally in need of oral care active agents. Think of, e.g., antiplaque agents, anti-tartar agents, anti-gingivitis agents, anti-bacterial agents, and others.

Such agents are generally administered from toothpastes and/or oral rinse liquids. Due to the typical environment of the oral cavity, e.g. having saliva present, a standard difficulty in the art is that active agents from toothpastes and oral rinses are quickly reducing in concentration after their application. Therefore they cannot protect the mouth for long times, and they need therefore to be applied several times daily.

WO 2008/135957 discloses a method for cleaning dental plaque biofilm from teeth wherein a liquid gellable composition is applied to the teeth. From the composition a gel layer is produced, wherein the gel layer adheres more strongly to the dental plaque bio film than the bio film adheres to the teeth. Ultimately the gel layer is removed from the teeth, and the dental plaque bio film along therewith, as the dental plaque bio film adheres to the gel layer.

The in situ formation of the gel can be realized by first administering a gellable liquid (e.g. based on chitosan), and then administering a second liquid (e.g. a sodium hydroxide solution) to bring about gelation. Other methods are disclosed in WO 2008/135957 as well, all involving the in situ formation of the gel. This has certain drawbacks in that not every user will be capable of correctly conducting the gel formation. It would therefore be desirable to provide a gel composition that is ready to use. This, however, brings about another challenge.

It will be interesting to provide oral care active agents in gel particles, particularly with sustained release of active agents therefrom. This would be an attractive solution to increase the concentration of oral care active agents for longer times. Gel particles generally have a low volume of solids (typically 1-2%) and therefore can contain a large volume of active formulation.

However, the actual delivery of active agents from particles administered into the oral cavity comes with challenges of its own. It would be desired to use jets or other types of devices to deliver the particles. As compared to conventionally applying the particles by rinsing, using an application device has the advantage that a substantially lower particle suspension volume is needed, and that the delivery can be targeted to the oral surface that needs the delivery the most, such as between the teeth, on the tongue or in the gingival pockets. Unfortunately, the inner linings of delivery devices have generally similar physico-chemical properties (surface charge and surface free energy) as the oral surfaces. Therefore, adhesive particles will also contaminate the insides of any delivery device, potentially leading to device failure and function degeneration.

### SUMMARY OF THE INVENTION

In order to better address the foregoing desires, the invention, in one aspect, concerns a system for providing an oral care composition, the system comprising a container holding a mucoadhesive oral care composition and an applicator adapted to introduce said oral care composition into the oral cavity of a subject, wherein the applicator comprises a wall that defines an inside of the applicator and wherein the surface of the wall at said inside is anti-adhesive to said oral care composition.

Preferably the oral care composition comprises gelled chitosan.

Preferably the oral care composition is selected from the group consisting of dentifrices, antiplaque compositions, anti-tartar compositions, anti-gingivitis compositions, anti-caries compositions, anti-bacterial compositions, compositions for periodontal treatment, and combinations thereof.

Preferably the the anti-adhesive surface is a conductive surface, and the system comprises a power source, the positive end of which is conductively connected to the conductive surface. Preferably the conductive connection between the power source and the surface can be switched on and off.

In another aspect, the invention presents an applicator for an oral care composition, said applicator comprising a wall that defines an inside of the applicator, wherein the surface of the wall at said inside is anti-adhesive to a mucoadhesive oral care composition.

In a further aspect, the invention provides an applicator for an oral care composition, said applicator comprising a wall that defines an inside of the applicator, wherein the surface of the wall at said inside is connected to a power source such that said surface can be rendered positively charged.

### BRIEF DESCRIPTION OF THE DRAWINGS

FIG. 1 is a magnified photograph (10x magnification) showing a polypropylene pipette tip when used for the administration of positively charged chitosan particles.

FIG. 2 is a photograph as in FIG. 1, the difference being that the inner surface of the pipette tip has been rendered positively charged.

FIG. 3. is a schematic drawing showing an embodiment of the system.

### DETAILED DESCRIPTION OF THE INVENTION

In a general sense, the invention is based on the judicious insight that applicators for oral care compositions can be rendered suitable for the application of mucoadhesive oral care compositions, by changing the inner surface of such applicator. The change serves to render the inner surface anti-adhesive, and preferably repellant, to the mucoadhesive oral care composition.

An “applicator” indicates any device that can be used in order to introduce an oral care composition into the oral cavity of a subject, such as a human. Typically, an applicator comprises a tubular part through which the composition can be forwarded into the oral cavity. It will be understood that such applicators are normally adapted for the delivery of oral care compositions into the mouth. Such a tubular part can be, e.g., a pipette, the tube and/or the needle of a syringe. In the event of a flexible tube, from which a composition can be applied by manual squeezing, the applicator for delivering the composition into the oral cavity will have to be provided with a narrow extension, such as a hose, or an extending nozzle. In the event that the container is a flexible tube, it will be understood that the regular capped orifice of such a tube, as in the case of toothpaste tubes, is not an applicator according to the invention. Rather, the orifice would have to be adapted as mentioned above by having a nozzle capable of delivering the composition into the mouth, e.g. by extending from the caped orifice.

In general, an applicator for this use will thus comprise a wall that defines an inside of the applicator. An applicator will generally be comprised in a delivery device, which either is identical with the applicator, or comprises further component parts, such as the plunger of a syringe, a generator for a water jet, a pump, and the like.

According to the invention, the surface of the wall at said inside is anti-adhesive to said oral care composition. In the present description said surface is hereinafter also indicated by the term “inner surface.” The inner surface thereby is the surface along which, at the inside of an applicator for an oral care composition, such a composition will have to pass when being applied to the oral cavity, using the applicator.

The inner surface can be the surface, at the inside of the applicator, of the material from which the wall of the applicator is made. E.g., in the event of a metal needle, the inner surface can be metal, and in the event of a polypropylene pipette, the inner surface can be polypropylene. The inner surface can also be a separate lining, applied on the inside of the applicator, such as a plastic layer provided inside a metal tube, against the wall of said tube. The inner surface can also be a coating, applied onto the inside surface of the wall. It is conceivable that all of the internal surfaces of the applicator are anti-adhesive to said composition. In an interesting embodiment, all of the internal surfaces that are designed to be in contact with the mucoadhesive composition, during storage and/or in use,

Where, in this description, the inner surface is said to be made of a material, this does not imply that the surface is necessarily made of only such material. It is intended to indicate, however, that the surface properties are determined, at least essentially so, by the properties of the material mentioned.

In one interesting embodiment, the inner surface of the applicator is rendered anti-adhesive per se by being made of a material having a low surface free energy, such as polytetrafluoroethylene (PTFE, known under the brand name of Teflon®). This can be particularly useful in the event that the adhesiveness of the oral care composition to surfaces is based on Van der Waals interactions or hydrophilic interactions. These are, e.g., copolymers of monoalkyl esters of poly (methyl vinyl ether/maleic acid), also traded under the name GANTREZ™. A reference in this respect is Molecules 2005, 10(1), 126-145; doi:10.3390/10010126, Bio adhesive Properties of Gantrez Nanoparticles by Juan M. Irache et al.

In another such embodiment, the inner surface can be provide with a coating that repels particles, such as coatings comprising polyethylene oxide (PEO) or polyethylene glycol (PEG) chains (also known as PEO/PEG brushes). Other anti-adhesive surface materials are known. These are generally used to generate so-called “non-stick” surfaces, which are engineered to reduce the ability of other materials to stick to it. Non-stick is often used to refer to surfaces coated with PTFE, but also to, e.g., silicone. For a use in applicators for mucoadhesive oral care compositions, PTFE is preferred for practical reasons.

A typical oral care composition for use in the system of the invention will comprise a gel as a carrier, and one or more oral care active agents. The composition can be directed to a specific use, such as a dedicated antibacterial composition, a dedicated whitening composition, or it can comprise a combination of active agents, such as present, e.g., in toothpaste or mouthwash. Particularly, the composition for use in the system of the invention can be considered as a visco-elastic mouthwash particle, suspended in a regular mouthwash.

The mucoadhesive oral care composition used in the invention preferably is a polymeric composition that is capable of forming solid particles, gel particles or capsules. Suitable polymers include polymethyl methacrylate beads, or methyl methacrylate copolymer beads. These are known, e.g., from the field of antibiotics, particularly as used in the treatment of hip infections. Preferred polymeric compositions are gels made of polysaccharide, particularly chitosan. Chitosan is known to be mucoadhesive.

Suitable mucoadhesive gels are described, e.g., in Fini et al., Pharmaceutics 2011, 3, 665-679. Other adhesive composition can also be used. Reference is made, e.g., to US 2007/258916, wherein dental compositions are described that are given a high viscosity for better adherence to teeth. Also, a phosphoric acid gel carrier is described that serves to further improve retention on dentin surfaces. It will be appreciated that the skilled person is well aware of various different oral care compositions designed to deliver active agents locally to the teeth, and which will benefit from the presence of magnetic material in accordance with the invention.

The oral care composition of the invention preferably comprises an oral care active agent selected from the group consisting of antiplaque agents, anti-tartar agents, anti-gingivitis agents, anti-bacterial agents, anti-caries agents, and combinations thereof.

The invention, in one aspect, concerns a system for providing an oral care composition, the system comprising a container holding a mucoadhesive oral care composition and an applicator comprising a wall that defines an inside of the applicator, wherein the surface of the wall at said inside is anti-adhesive to said oral care composition. In view of the foregoing, in one embodiment the system comprises an oral care composition the adhesiveness of which is based on Van Der Waals interaction or on hydrophilic interaction, and the applicator comprises an inner surface made of a non-stick material, preferably polytetrafluoroethylene.

The system of the invention preferably further comprises a dental appliance for cleaning teeth, selected from the group consisting of electric toothbrushes, electric flossing devices, and combinations thereof. Such dental appliances can be provided for various functions. This typically refers to a toothbrush, preferably an electrical toothbrush, more preferably a sonic power toothbrush having a vibrating brushhead.

An electric flossing device, as is possibly comprised in the system of the invention, refers to such devices that serve to clean the interdental spaces generally by spraying air, by spraying liquid, or a combination thereof. In the system of the invention, such a device can also be of use in order to direct the oral care composition to the desired location in the oral cavity by the force of spraying.

It is to be understood that the system can comprise its various parts as separate components, not packaged or provided together.

Particularly, the container holding the mucoadhesive oral care composition can well be provided as a separate entity, e.g., in the form of a bottle or tube holding the composition (comparable to a bottle of mouthwash or a toothpaste tube). The container can also be attached to the delivery device, particularly as a cartridge adapted for such an attachment, e.g. to an electric toothbrush, flossing device or an oral irrigator, such as a Philips Sonicare AirFloss or Philips Sonicare Toothbrush, with a delivery pump.

In a preferred embodiment, the mucoadhesive oral care composition is positively charged, such as in the event of the aforementioned gellable particles based on chitosan. These compositions are advantageous per se, but also cooperate, synergistically, with a preferred method to secure the anti-adhesiveness of the inner surface of an applicator for such a composition.

To this end, the inner surface of the applicator is made to be positively charged. By nature, the positive charge of the inner surface of the applicator will repel the positively charged composition.

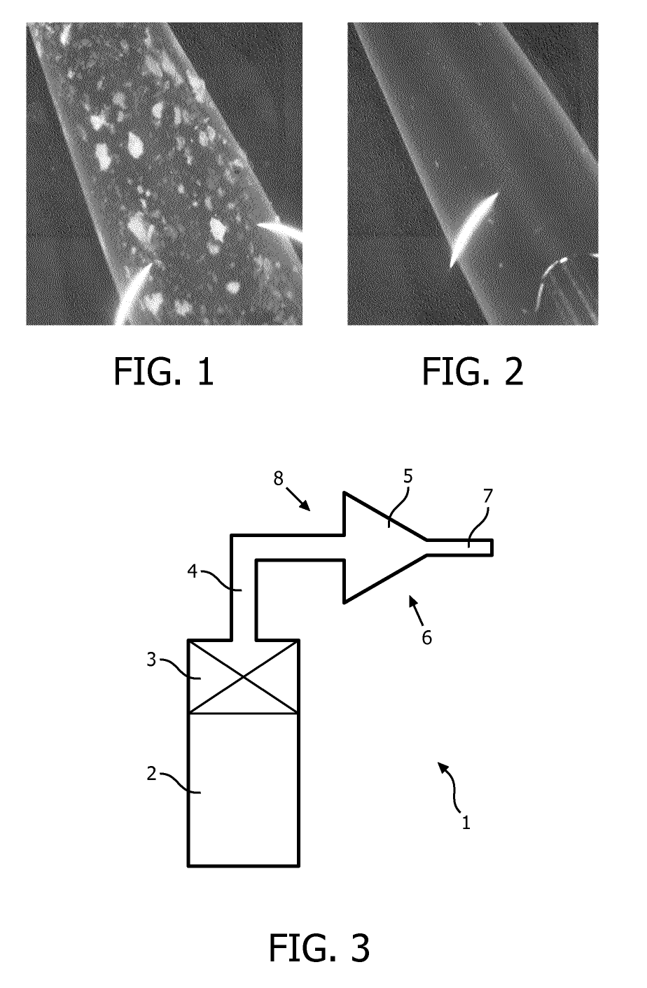

This is shown in FIG. 1 and FIG. 2. In FIG. 1, a chitosan-based (positively charged) composition is passed through the polypropylene tip of a pipette. As shown by the white spots in the photograph, a considerable amount of particles is retained on the inner surface of the pipette tip, as a contamination thereof. In FIG. 2, the same composition is passed through the same type of pipette, the difference being that a positive charge is applied to the inner surface of the pipette tip. As seen in the photograph, hardly or no contamination with the composition occurs. The positive charge is applied by coating the surface with a thin layer of polylysine (which is a positively charged polymer). Other positively charged coatings are available as well, e.g. based on acrylate polymers (particularly copolymers) which are versatile materials, and allow the skilled person to provide charged polymers that can be stronger, cheaper, or both than polylysine. E.g., polymers of methyl methacrylate with a percentage (e.g. 5-50%, preferably 10-20%, typically 15%) of methacrylic acid (PMMA/MAA) or trimethyl amino ethyl methacrylate chloride (PMMA/TMAEMA-Cl). See, e.g., Gottenbos et al., Journal of Materials Science: Materials in Medicine, 10, 1999, 853-855.

The present invention is the first to realize the usefulness of applying a positive charge to the inner surface of an applicator for a mucoadhesive oral care composition. In this respect, the invention also pertains to an applicator for an oral care composition. The applicator according to the invention comprises a wall that defines an inside of the applicator, wherein the surface of the wall at said inside is positively charged.

The positive charge can be provided by using positively charged bulk materials, such as co-polymers of methyl methacrylate and trimethylaminoethyl methacrylate chloride, or any other material possessing an amine, quaternary ammonium or other positively charged group. Alternatively, only the surface is coated with such a positively charged material. This is advantageous, as it presents an economically more beneficial solution, and the resulting applicators are more durable if they themselves are made from standard plastics, such as polypropylene, rather than entirely of the aforementioned polymers.

In a further interesting embodiment, metal or other conducting materials are used, either as the bulk material for the applicator wall, or in a coating applied onto the applicator wall. This presents the advantage that then the inner surface of the applicator wall can be become charged positively by conductively connecting it to the positive end of a power source (e.g. a battery). This provides the advantage that the charge can be tuned externally, and can be turned on and off. This is of particular benefit for applicators that are used with electric dental care appliances, such as electric toothbrushes, electric flossing devices, or electric oral irrigators, as such devices have power available as a standard feature. Also, it would enable using the same applicator for delivering different compositions or materials into the oral cavity. E.g., the same hollow needle can then be used for spraying water or dental rinse fluids into the oral cavity, and for applying a fluid, gellable composition that is mucoadhesive.

As an alternative to switching the power on or off, the system of the invention can also be provide with a conductive connection between the power source and the inner surface of the applicator, whereby said connection can be switched on and off.

Thus, in an interesting embodiment, the system of the invention comprises a power module and one or more dental appliance heads that can be removably attached to said power module, wherein one of the dental appliance heads is an applicator in accordance with the present invention, as described hereinbefore.

While the invention has been illustrated and described in detail in the drawings and foregoing description, such illustration and description are to be considered illustrative or exemplary and not restrictive; the invention is not limited to the disclosed embodiments. For example, it is possible to operate the invention in an embodiment wherein only part of the applicator is provided with an anti-adhesive inner surface, e.g. the tube of a pipette is anti-adhesive, and separate (not necessarily anti-adhesive) pipette tips are provided for one-time use.

FIG. 3 schematically shows a system 1 for providing an oral care composition according to the present invention. The system comprises a container 2 that holds a mucoadhesive oral care composition and an applicator 8. The applicator comprises a nozzle 6 and a tube 4 that connects the container 2 with the nozzle 6. The tube 4 can be both rigid or flexible. A pump 3 is provided for the delivery of the oral care composition to the applicator 8. An inside-wall 5 of the applicator is anti-adhesive to the oral care composition. The nozzle 6 comprises a needle 7. The applicator 8 is detachable from the container 2 and can be part of an electric toothbrush, an electric flossing device or an electric oral irrigator.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain features of the invention are recited in mutually different dependent claims does not indicate that a combination of these features cannot be used to advantage. Any reference signs in the claims should not be construed as limiting the scope. In sum, we hereby disclose a new system for the application of mucoadhesive oral care compositions. The system comprises an applicator, the inner surface of which is made anti-adhesive. Particularly, said inner surface is rendered positively charged, so as to be anti-adhesive for positively charged mucoadhesive oral care compositions, such as those based on chitosan.

1. A system for providing an oral care composition, the system comprising a container holding a mucoadhesive oral care composition and an applicator adapted to introduce said oral care composition into the oral cavity of a subject, wherein the applicator comprises a wall that defines an inside of the applicator and wherein the surface of the wall at said inside is anti-adhesive to said oral care composition.

2. A system according to claim 1, wherein the oral care composition is a gel, preferably in the form of particles.

3. An applicator adapted to introduce a mucoadhesive oral care composition into the oral cavity of a subject, said applicator comprising a wall that defines an inside of the applicator, wherein the surface of the wall at said inside is anti-adhesive to the mucoadhesive oral care composition.

4. An applicator according to claim 3, wherein the anti-adhesive surface is made of polytetrafluoroethylene.

5. An applicator according to claim 3, wherein the anti-adhesive surface is positively charged.

6. An applicator according to claim 3 wherein the surface of the wall at said inside is conductive, and is conductively connected to a power source such that said surface can be rendered positively charged.

7. An applicator according to claim 3, wherein the applicator is attachable to an electric toothbrush, an electric flossing device or an electric oral irrigator.

8. An applicator according to claim 3, wherein the applicator is an electric toothbrush, an electric flossing device or an electric oral irrigator.

9. An applicator according to claim 3, wherein the applicator comprises a pump adapted for the delivery of the mucoadhesive oral care composition.

10. An applicator according to any claim 3, wherein the applicator comprises a nozzle capable of delivering the mucoadhesive oral care composition into the mouth.

11. An applicator according to claim 10, wherein the nozzle comprises a hollow needle.

12. An applicator according to claim 3, wherein all of the internal surfaces of the applicator that are designed to be in contact with the mucoadhesive composition, during storage and/or in use, are anti-adhesive to the mucoadhesive oral care composition.

13. System for providing an oral care composition, the system comprising a container holding a mucoadhesive oral care composition and an applicator adapted to introduce said oral care composition into the oral cavity of a subject according to claim 3.
